(19) United States
(12) Patent Application Publication
(10) Pub. No.: US 2019/0051383 A1
(43) Pub. Date: Feb. 14, 2019

(54) INTELLIGENT SEPSIS ALERT

(71) Applicant: WAYNE STATE UNIVERSITY, DETROIT, MI (US)

(72) Inventors: Robert Sherwin, Northville, MI (US); Hao Ying, Novi, MI (US)

(73) Assignee: WAYNE STATE UNIVERSITY, Detroit, MI (US)

(21) Appl. No.: 16/059,254

(22) Filed: Aug. 9, 2018

Related U.S. Application Data
(60) Provisional application No. 62/543,038, filed on Aug. 9, 2017.

(51) Int. Cl. G16H 10/60; G16H 10/40; G16H 80/00

(52) U.S. Cl. CPC G16H 10/60; G16H 80/00; G16H 10/40

(57) ABSTRACT

A system for determining a likelihood of current or near-future occurrence of sepsis in a patient analyzes patient information through applying a computational decision-making algorithm that is linked to patient category. If the result of the analysis satisfies the criteria of the algorithm, an alert is transmitted to a caregiver. The results of the analysis are stored in the system, and the stored results are periodically and automatically analyzed relative to false positives, false negatives, and correct decisions as part of the alert system operation. The algorithm and its related components are automatically modified to improve alert accuracy in subsequent applications of the system.

### CROSS-REFERENCE TO RELATED APPLICATIONS

This application claims priority to U.S. Provisional Application No. 62/543,038, which was filed Aug. 9, 2017, and is incorporated by reference in its entirety.

### TECHNICAL FIELD

The present application relates to a computational system for providing a medical alert. More particularly, the system provides a clinical-decision making analysis of a likelihood that a patient in a healthcare facility either has sepsis or will have sepsis in the near future.

### BACKGROUND

This statement in this section merely provides background information related to the present disclosure and may not constitute prior art. Sepsis is common and a potentially life-threatening complication of an infection. Sepsis occurs when chemicals released into the bloodstream to fight the infection trigger inflammatory responses throughout the body. This inflammation can trigger a cascade of changes that can damage multiple organ systems, causing them to fail. It represents a healthcare epidemic that hospitalizes over 1.6 million people annually in the U.S. alone.

Sepsis represents a host's dysfunctional response to infection and includes a spectrum of disease severity from mild (sepsis), moderate (severe sepsis) to most severe (septic shock). It is the most common cause of shock and the most common cause of death in non-cardiac intensive care units. Though the diagnostic criteria for sepsis appear relatively straightforward, sepsis is actually a diagnostic challenge.

We have discovered that physicians commonly under-detect sepsis. Early sepsis recognition is challenging due to the heterogeneity of patients who may manifest a wide array of clinical presentations. Another major contributing factor is that many septic patients may initially present without organ dysfunction or evidence of shock and only develop these later during their stay in healthcare facilities.

The above information disclosed in this Background section is only for enhancement of understanding of the background of the present disclosure and therefore it may contain information that does not form the prior art that is already known to a person of ordinary skill in the art.

### SUMMARY

It is an object of the present disclosure to determine a likelihood of current or near-future occurrence of sepsis in a patient and provide an alert to a healthcare provider if the likelihood is high.

This objective is achieved by providing a system determining a likelihood of a current or near-future occurrence of sepsis in a patient, the system comprising a computing device having a processor and a non-transitory computer readable medium having instructions stored thereon that, when executed by the processor, cause the computing device to perform the steps of: receiving patient information at an electronic medical record (EMR) database; outputting the patient information from the EMR to a sepsis identifier; classifying the patient into a category based on at least a portion of the patient information; choosing a component comprising at least one of variables, parameter values, and alert criteria based on the category of the patient; analyzing the patient information with the selected component or components based on the category of the patient and determining an output of the analysis of the patient information; determining a result whether the output of the analysis satisfies a criteria to generate an alert; and providing the determined result.

The system may further include receiving the previously determined result stored in the EMR; receiving an actual result determined independently from the system; comparing the determined result to the actual result; and in response to comparing the determined result to the actual result, modifying the instructions to determine a different result of the analysis in subsequent applications of the system.

In accordance with another embodiment of the present disclosure, in the system, the determined output is the likelihood of a current or near-future occurrence of sepsis in the patient for informing the alert to a caregiver, and the likelihood output is stored into the EMR. If the likelihood of sepsis in the patient exceeds an established criterion, the system determines a yes result and provides the alert via a communication network to the caregiver, and provides the alert result to the EMR. If the likelihood of sepsis in the patient does not exceed an established criterion, the system does not send the alert via a communication network to the caregiver, and provides the no alert result to the EMR.

In accordance with another embodiment of the present disclosure, the system is an intelligent sepsis alert system including the EMR, the sepsis identifier, a learning and optimizing module and a communication network. The learning and optimizing module is configured to minimize repeating historical alert mistake by modifying the components in the sepsis identifier. The module includes a historical decision analyzer, an alert performance analyzer and an identifier modifier. Furthermore, the sepsis identifier is optimized autonomously through joint operations of the learning and optimizing module.

In accordance with another embodiment of the present disclosure, the EMR includes medical records, a laboratory database, an administration database, a pharmacy database and a historical alert decision database.

In accordance with another embodiment of the present disclosure, the sepsis identifier includes a patient categorizer, a sepsis decision maker and a component selector.

Further benefits of the proposed system will become evident from the following description of preferred embodiments shown in the drawings. The drawings are provided herewith solely for illustrative purposes and are not intended to limit the scope of the present invention.

### DRAWINGS

In order that the disclosure may be well understood, there will now be described various forms thereof, given by way of example, reference being made to the accompanying drawings, in which:

FIG. 1 shows a schematic view of a computer device for implementing the method described herein; and

FIG. 2 shows a block diagram of real-time computer-implemented intelligent sepsis alert system.

The drawings described herein are for illustration purposes only and are not intended to limit the scope of the present disclosure in any way.

### DETAILED DESCRIPTION

The following description is merely exemplary in nature and is in no way intended to limit the present disclosure or its application or uses. It should be understood that throughout the drawings, corresponding reference numerals indicate like or corresponding parts and features.

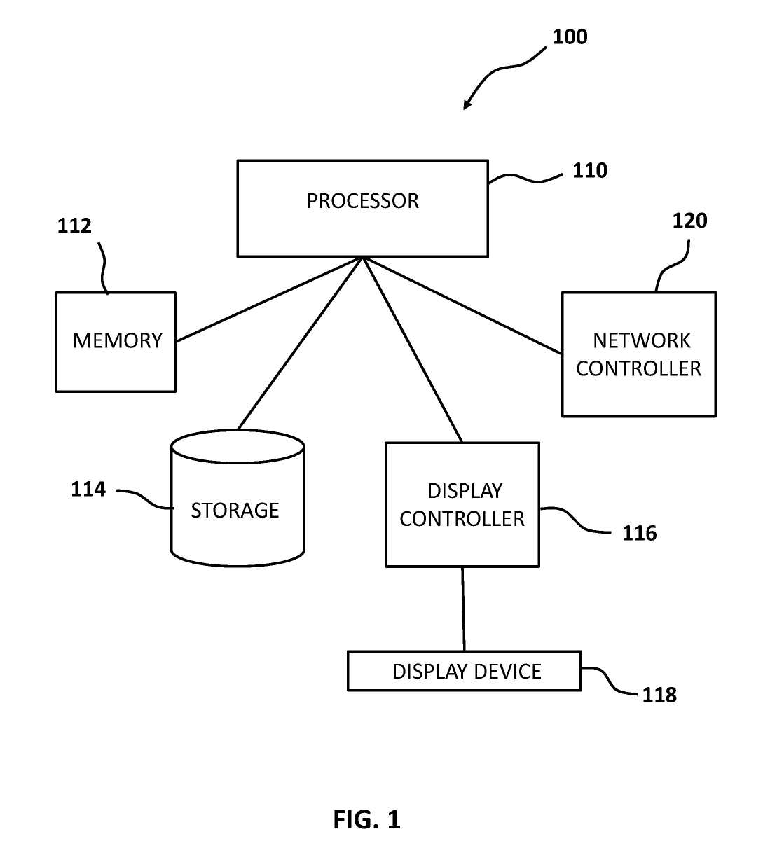

An exemplary operating system suitable for implementing embodiments of the present disclosure is described below. Referring to FIG. 1, an exemplary operating system for implementing embodiments of the present disclosure is shown and designated generally as a computing device 100. The computing device 100 is an example of a suitable computing environment and is not intended to suggest any limitation as to the scope of use or functionality of embodiment of the present disclosure.

Embodiments of the present disclosure may be described in the general context of computer code or machine-usable instructions, including computer-executable instructions such as program components, being executed by a computer or other machine, such as a personal data assistant or other handled device. Generally, program components including routines, programs, objects, components, data structures, and the like refer to code that performs particular tasks, or implementations particular abstract data types. Embodiments of the present disclosure may be practiced in a variety of system configurations, including handheld devices, consumer electronics, general-purpose computers, and specialty computing devices, etc. Embodiments of the present disclosure may also be practiced in distributed computing environments where tasks are performed by remote-processing devices that are linked through a communication network.

As shown in FIG. 1, the computing device 100 includes a processor 110 for executing instructions such as methods described herein. The instructions may be stored in a non-transitory computer readable medium such as memory 112 or a storage device 114, for example a disk drive, CD, or DVD. The computer device 100 may include a display controller 116 responsive to instructions to generate a textual or graphical display on a display device 118, for example a computer monitor or a handheld device. In addition, the processor 110 may communicate with a network controller 120 to communicate data or instructions to other systems, for example other general computer systems or servers. The network controller 120 may communicate over Ethernet or other known protocols to distribute processing or provide remote access to information over a variety of network topologies, including local area network, wide area networks, the internet, or other commonly used network topologies.

As described above, furthermore, the executing instructions may be stored in the computer readable medium. The term “computer readable medium” includes a single medium or multiple media, such as a centralized or distributed database, and/or associated caches and servers that store one or more sets of instructions. The term “computer readable medium” shall also include any medium that is capable of storing, encoding, or carrying a set of instructions for execution by the processor 110 or that cause a computer system to perform any one or more of the methods or operations disclosed herein.

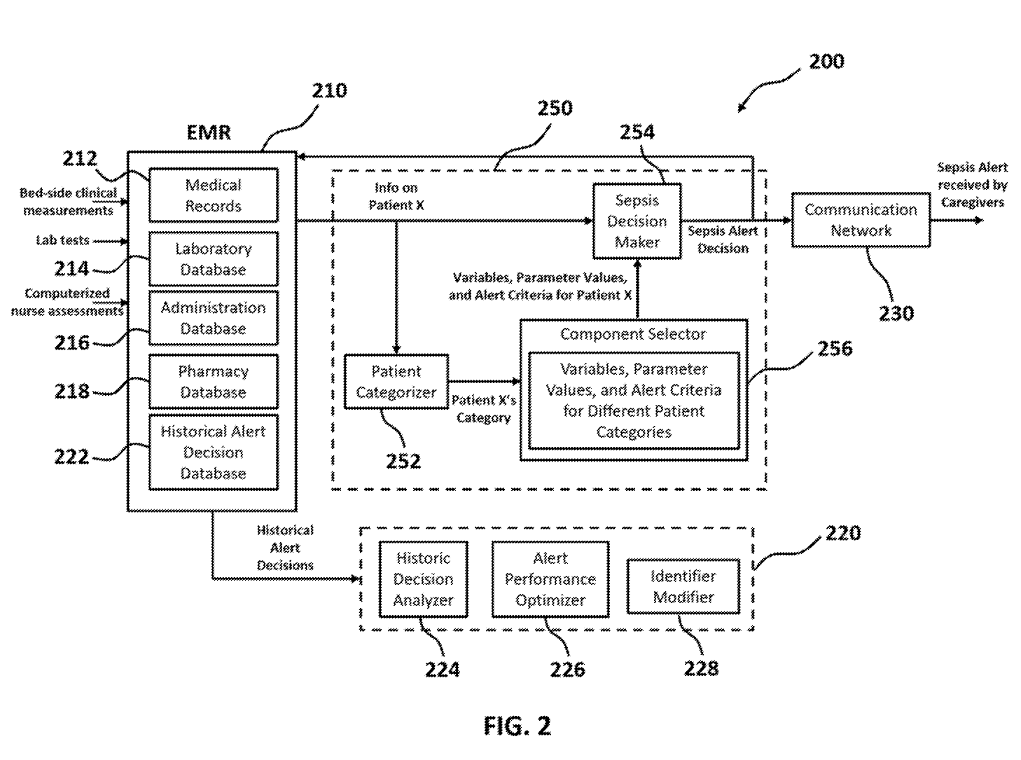

Referring now to FIG. 2, a block diagram of an exemplary system is shown for a computer-implemented real-time system and method for continuously monitoring a patient under care in any healthcare facility (such as, but not limited to, a hospital), in accordance with an embodiment of the present disclosure. The exemplary system is an intelligent sepsis alert system 200. The system 200 signals an electronic sepsis alert over a communication network 230 to a caregiver in the healthcare facility when the patient either exhibits an initial sign of sepsis or is predicted to exhibit sign of sepsis in the near future. As shown in FIG. 2, the sepsis alert system 200 includes an Electronic Medical Record (EMR) 210, a Sepsis Identifier 250, a Learning and Optimizing Module 220, and the communication network 230 as sub-systems.

The system 200 is capable of constantly improving its alert performance in an autonomous manner by an identifier modifier 228 in the learning and optimizing module 220 to modify various components of the sepsis identifier 250. The sepsis alert system 200 takes advantage of fuzzy system technology for representing and processing imprecise expert knowledge and experience, and evolutionary computing technology for optimizing alert performance. It also takes advantage of machine learning technology for self-learning, and intelligent system technology for autonomous correcting and improving behaviors. These combined technologies lead to earlier and more reliable sepsis alert to caregivers. Accordingly, the system 200 improves the sepsis alert by optimizing its performance accuracy while minimizing false positives and false negatives.

As shown in FIG. 2, the sepsis alert system 200 is collecting and utilizing the information from the electronic medical record (EMR) 210 of the patient under care in the healthcare facility, and the information about real-time status of the patient to determine if and when to issue alert. The EMR 210 has various databases such as medical records 212, a laboratory database 214, an administration database 216, a pharmacy database 218, and a historical alert decision database 222.

The information is input into EMR 210 during the course of the patient's stay in the healthcare facility. For example, demographics, vital signs, bed-side clinical measurements, lab-tests and computerized nurse assessments may be entered into the EMR 210. During the patient's staying in the healthcare facility, the caregiver in each department such as a laboratory for the lab-tests, a pharmacy department for the patient's medicine information including a pharmacy database and an administration office for the patient's demographics, etc. enters the patient's condition into the EMR 210. By inputting the data, the patient information is sent to the medical record server where it is stored.

As shown in FIG. 2, the EMR 210 communicates with the sepsis identifier 250 as a part of the sepsis alert system 200. From the EMR 210 storing all information of the patient during staying in the healthcare facility, the information is sent to a patient categorizer 252 and a sepsis decision maker 254 in the sepsis identifier 250. In addition, the sepsis identifier 250 further includes a component selector 256 that chooses variables, parameter values, and alert criteria for different patient categories.

The patient categorizer 252 in the sepsis identifier 250 performs personalized monitoring. The patient categorizer 252 categorizes all patients in a diverse population into groups. The risk of sepsis and its complications are generally higher or lower depending on specific clinical and demographic characteristic of the patient.

The patient categorizer 252 determines and outputs a category or group for the patient by applying a unique algorithm that partitions patient into separate unique categories. Each category may have its own variables, parameter values, and alert criteria for real-time sepsis identification. For the particular patient, information on components chosen by the component selector 256 may be sent to the sepsis decision maker 254, which analyses the patient information with the help of the component information. The sepsis decision maker 254 is a computational algorithm that may be implemented as a rule-based system resulted from various decision tree techniques (including boosted tress and bagged trees). The sepsis decision maker 254 may also be implemented as an algorithm of such technique as support vector machine, neural network, random forest, quadratic discriminant, K nearest neighbor, or logistic regression.

For example, the sepsis decision maker 254 may determine a sepsis likelihood for a particular patient, and if the likelihood satisfies the criteria of the logic or algorithm (for example, the likelihood exceeds an established threshold), an alert will be made by the sepsis identifier 250. The sepsis decision maker 254 determines whether to generate a sepsis alert. The final decision may be in the form of a yes/no decision. If the final result of the sepsis decision maker 254 is a yes, the sepsis identifier 250 generates an alert and sends the alert to the caregiver via the communication network 230. The generated sepsis alert signal is received by the caregiver in the healthcare facility. If the likelihood does not exceed the established criterion, no alert will be made by the sepsis identifier 250 and the sepsis identifier 250 provides the no alert result to the EMR 210.

In the sepsis identifier 250, the setting for each patient category can be different, which will be optimized autonomously for that group or category, through joint operations of the learning and optimizing module 220 to achieve better alert performance over time. For example, the sepsis identifier 250 directly and automatically communicates with the EMR 210 and receives new information when the patient's medical record is updated. Accordingly, the learning and optimizing module 220 adds, if needed, new categories and other related elements such as variables, parameter values and/or alert criteria to be used by the component selector 256. The variables involved include, but are not limited to age, respiratory rate, heart rate, systolic pressure, temperature, white blood cells, lactate, renal condition, and HIV status.

In FIG. 2, the sepsis alert system 200 is further configured to minimize repeating historical alert mistakes. Each instance of the alert system's output is stored in the sepsis alert system 200. The result of the sepsis decision maker 254 is output into the EMR 210 and stored in the patient's historical alert decision database 222 in the EMR 210. The stored results are sepsis likelihood, and also whether the alert is issued for the patient. The information stored in the historical alert decision database 222 is sent to the learning and optimizing module 220.

As shown in FIG. 2, the learning and optimizing module 220 includes a historic decision analyzer 224, an alert performance optimizer 226, and the identifier modifier 228. As the number of patients processed by the system 200 grow, the module 220 periodically and autonomously analyzes the alert decisions of the system 200 whether they are correct or incorrect on all the historic patients via the historic decision analyzer 224. The module 220 modifies components in the sepsis decision maker 254, the patient categorizer 252, and/or the component selector 256 through the identifier modifier 228 for minimizing the same mistakes on future patients, if warrantied.

For example, in the event of a false positive (where an alert is sent but the patient does not have sepsis) or a false negative (where an alert is not sent but the patient is determined otherwise to have sepsis), the result of the false positive and/or false negative may be input into the historical alert decision database 222. Correct decisions may also be provided to the module 220 from the historical alert decision database 222. These results of correct or false decisions (historical alert decision) are sent to the learning and optimizing module 220 and are analyzed by the historic decision analyzer 224 with the result being sent to the alert performance optimizer 226. The optimizer 226 determines which components of the sepsis identifier 250 should be modified. The identifier modifier 228 carries out the needed modification to the sepsis decision maker 254, the patient categorizer 252, and/or the component selector 256 of the sepsis identifier 250 for alert performance improvement in patients.

Furthermore, the above features result in earlier and more accurate alerts while simultaneously minimizing the number of false positives and false negatives. A false positive occurs when a method is in error and alerts a caregiver that a patient has sepsis when in fact the patient does not. These errors create needless effort and create dangerous healthcare environment.

Accordingly, the sepsis alert system 200 includes the information and historical alert decisions of each patient that is monitored within the system 200. Additional iteration of the system will provide further information that may result in further modifications, thereby providing an autonomous alert system that improves its performance over time.

The foregoing description of various forms of the invention has been presented for purposes of illustration and description. It is not intended to be exhaustive or to limit the invention to the precise forms disclosed. Numerous modifications or variations are possible in light of the above teachings. The forms discussed were chosen and described to provide the best illustration of the principles of the invention and its practical application to thereby enable one of ordinary skill in the art to utilize the invention in various forms and with various modifications as are suited to the particular use contemplated. All such modifications and variations are within the scope of the invention as determined by the appended claims when interpreted in accordance with the breadth to which they are fairly, legally, and equitably entitled.

What is claimed is:

1. A system for determining a likelihood of a current or near-future occurrence of sepsis in a patient, the system comprising a computing device having a processor and a non-transitory computer readable medium having instructions stored thereon that, when executed by the processor, cause the computing device to perform the steps of:
receiving patient information at an electronic medical record (EMR) database;
outputting the patient information from the EMR to a sepsis identifier;
classifying the patient into a category based on at least a portion of the patient information;
choosing a component comprising at least one of variables, parameter values, and alert criteria based on the category of the patient;
analyzing the patient information with the selected component or components based on the category of the patient and determining an output of the analysis of the patient information;
determining a result whether the output of the analysis satisfies a criteria to generate an alert; and
providing the determined result.

2. The system of claim 1 further comprising:
receiving the previously determined result stored in the EMR;
receiving an actual result determined independently from the system;
comparing the determined result to the actual result; and
in response to comparing the determined result to the actual result, modifying the instructions to determine a different result of the analysis in subsequent applications of the system.

3. The system of claim 1, wherein the determined output is the likelihood of a current or near-future occurrence of sepsis in the patient for informing the alert to a caregiver, and the likelihood output is stored into the EMR.

4. The system of claim 3, wherein if the likelihood of sepsis in the patient exceeds an established criterion, the system determines a yes result and provides the alert via a communication network to the caregiver, and provides the alert result to the EMR.

5. The system of claim 3, wherein if the likelihood of sepsis in the patient does not exceed an established criterion, the system does not send the alert via a communication network to the caregiver, and provides the no alert result to the EMR.

6. The system of claim 1, wherein the system is an intelligent sepsis alert system including the EMR, the sepsis identifier, a learning and optimizing module and a communication network.

7. The system of claim 6, wherein the learning and optimizing module is configured to minimize repeating historical alert mistake by modifying the components in the sepsis identifier.

8. The system of claim 6, wherein the learning and optimizing module includes a historical decision analyzer, an alert performance analyzer and an identifier modifier.

9. The system of claim 6, wherein the sepsis identifier is optimized autonomously through joint operations of the learning and optimizing module.

10. The system of claim 1, wherein the EMR includes medical records, a laboratory database, an administration database, a pharmacy database and a historical alert decision database.

11. The system of claim 1, wherein the sepsis identifier includes a patient categorizer, a component selector, and a sepsis decision maker.
